(19) United States
(12) Patent Application Publication
(10) Pub. No.: US 2007/0219616 A1
(43) Pub. Date: Sep. 20, 2007

(54) Notched catheter

(76) Inventors: D. Bruce Modesitt, San Carlos, CA (US); George D. Hermann, Portola Valley, CA (US); Jonathan M. Olson, San Jose, CA (US); Marshall Tsuruda, San Jose, CA (US); Peter E. Rosario, Fremont, CA (US); Donald J. Cabaluna, Foster City, CA (US)

Correspondence Address: ADAMS AND REESE LLP, 4400 ONE HOUSTON CENTER, 1221 MCKINNEY, HOUSTON, TX, 77010, US

(21) Appl. No.: 11/748,913

(22) Filed: May 15, 2007

Related U.S. Application Data
(62) Division of application No. 11/175,111, filed on Jul. 5, 2005.
Application No. 11/175,111 is a continuation-in-part of application No. 11/018,563, filed on Dec. 20, 2004.

(51) Int. Cl. A61F 2/06

(52) U.S. Cl. 623/1.11

(57) ABSTRACT

A notched catheter has a lumen and a notched outer surface intersecting the lumen with a filament-containing layer adjacent to the outer surface. A part of the filament-containing layer is capturable between the notched surface portion and an elongate element extendable through the lumen.

### CROSS-REFERENCE TO RELATED APPLICATIONS

This Divisional U.S. Patent Application, filed under 37 C.F.R. § 1.53(b) and 35 U.S.C. § 121, claims the benefit of priority under 35 U.S.C. § 120 of U.S. patent application Ser. No. 11/175,111 (attorney docket no. 14558-40, filed on Jul. 5, 2005), which is related to U.S. patent application Ser. No. 11/175,112 (attorney docket no. 14558-41, filed Jul. 5, 2005), both of which are Continuations in Part of U.S. patent application Ser. No. 11/018,563 (attorney docket no. 14558-37, filed Dec. 20, 2004), each of which is hereby incorporated by reference as to its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

Stents, covered stents and other endoluminal prostheses are often useful for placement in various hollow body structures, such as blood vessels, including coronary arteries, iliac arteries and femoro-popliteal arteries, the ureter, urethra, bronchus, biliary tract, gastrointestinal tract and the like, for the treatment of conditions which may benefit from the introduction of a reinforcing or protective structure and/or the introduction of a therapeutic agent within the body lumen. The prostheses will typically be placed endoluminally. As used herein, “endoluminally” will mean placement by percutaneous or cutdown procedures, wherein the prosthesis is transluminally advanced through the body lumen from a remote location to a target site in the lumen. In vascular procedures, the prostheses will typically be introduced “endovascularly” using a catheter over a guide wire under fluoroscopic, or other imaging system, guidance. The catheters and guide wires may be introduced through conventional access sites to the vascular system, such as through the femoral artery, or brachial and subclavian arteries, for access to the target site.

An endoluminal prosthesis typically comprises at least one radially expansible, usually cylindrical, body segment. By “radially expansible,” it is meant that the body segment can be converted from a small diameter configuration (used for endoluminal placement) to a radially expanded, usually cylindrical, configuration, which is achieved when the prosthesis is implanted at the desired target site. The prosthesis may be non-resilient, e.g., malleable, thus requiring the application of an internal force to expand it at the target site. Typically, the expansive force can be provided by a balloon catheter, such as an angioplasty balloon for vascular procedures. Alternatively, the prosthesis can be self-expanding. Such self-expanding structures may be provided by a temperature-sensitive superelastic material, such as Nitinol, which naturally assumes a radially expanded condition once an appropriate temperature has been reached. The appropriate temperature can be, for example, a temperature slightly below normal body temperature; if the appropriate temperature is above normal body temperature, some method of heating the structure must be used. Another type of self-expanding structure uses resilient material, such as a stainless steel or superelastic alloy, such as Nitinol, and forming the body segment so that it possesses its desired, radially-expanded diameter when it is unconstrained, e.g., released from radially constraining forces of a sheath. To remain anchored in the body lumen, the prosthesis will remain partially constrained by the lumen. The self-expanding prosthesis can be delivered in its radially constrained configuration, e.g. by placing the prosthesis within a delivery sheath or tube and retracting the sheath at the target site. Such general aspects of construction and delivery modalities are well known in the art.

The dimensions of a typical endoluminal prosthesis will depend on its intended use. Typically, the prosthesis will have a length in the range from 0.5 cm to 25 cm, usually being from about 0.8 cm to 10 cm, for vascular applications. The small (radially collapsed) diameter of cylindrical prostheses will usually be in the range from about 1 mm to 10 mm, more usually being in the range from 1.5 mm to 6 mm for vascular applications. The expanded diameter will usually be in the range from about 2 mm to 50 mm, preferably being in the range from about 3 mm to 15 mm for vascular applications and from about 25 mm to 45 mm for aortic applications.

One type of endoluminal prosthesis includes both a stent component and a covering component. These endoluminal prostheses are often called stent grafts or covered stents. A covered stent is typically introduced using a catheter with both the stent and covering in contracted, reduced-diameter states. Once at the target site, the stent and covering are expanded. After expansion, the catheter is withdrawn from the vessel leaving the covered stent at the target site. Coverings may be made of, for example, PTFE, ePTFE or Dacron.RTM. polyester.

Grafts are used within the body for various reasons; such as to repair damaged or diseased portions of blood vessels such as may be caused by injury, disease, or an aneurysm. It has been found effective to introduce pores into the walls of the graft to provide ingrowth of tissue onto the walls of the graft. With larger diameter grafts, woven graft material is often used. In small and large diameter vessels, porous fluoropolymers, such as ePTFE, have been found useful.

Coil-type stents can be wound about the catheter shaft in torqued compression for deployment. The coil-type stent can be maintained in this torqued compression condition by securing the ends of the coil-type stent in position on a catheter shaft. The ends are released by, for example, pulling on wires once at the target site. See, for example, U.S. Pat. Nos. 5,372,600 and 5,476,505. Alternatively, the endoluminal prosthesis can be maintained in its reduced-diameter condition by a sleeve; the sleeve can be selectively retracted to release the prosthesis. A third approach uses a balloon to expand the prosthesis at the target site. The stent is typically extended past its elastic limit so that it remains in its expanded state after the balloon is deflated and removed. One balloon expandable stent is the Palmaz-Schatz stent available from the Cordis Division of Johnson & Johnson. Stents are also available from Medtronic AVE of Santa Rosa, Calif. and Guidant Corporation of Indianapolis, Ind. A controlled release catheter assembly, such as disclosed in U.S. Pat. Nos. 6,238,430 and 6,248,122, may also be used to deploy a coiled prosthesis. See also U.S. Pat. No. 6,572,643.

The following patents may be of interest. U.S. Pat. No. 6,660,032 issued Dec. 9, 2003; U.S. Pat. No. 6,645,237 issued Nov. 11, 2003; U.S. Pat. No. 6,572,648 issued Jun. 3, 2003; U.S. Pat. No. 6,514,285 issued Feb. 4, 2003; U.S. Pat. No. 6,371,979 issued Apr. 16, 2002; U.S. Pat. No. 5,824,053 issued Oct. 20, 1998; U.S. Pat. No. 5,772,668 issued Jun. 30, 1998; U.S. Pat. No. 5,443,500 issued Aug. 22, 1995; U.S. Pat. No. 4,760,849 issued Aug. 2, 1988; and U.S. Pat. No. 4,553,545 issued Nov. 19, 1985. See also PCT Publication Number WO 94/22379 published Oct. 13, 1994; and PCT Publication Number WO 94/16629 published Aug. 4, 1994.

### BRIEF SUMMARY OF THE INVENTION

The invention is directed to a notched catheter comprising a catheter body having an outer surface and a longitudinally extending lumen. The outer surface has a generally cylindrical surface portion and a notched surface portion, the notched surface portion extending from the generally cylindrical surface portion to intersect the lumen. A filament-containing layer is adjacent to the generally cylindrical surface portion and the notched surface portion. A portion of the filament-containing layer is capturable between the notched surface portion and an elongate element extendable through the lumen.

Various features and advantages of the invention will appear from the following description in which the preferred embodiments have been set forth in detail in conjunction with the accompanying drawings.

### BRIEF DESCRIPTION OF THE DRAWINGS

FIG. 1 is an overall view of a coiled stent delivery assembly made according to the invention;

FIG. 1A is an overall view of release wire assembly of FIG. 1;

FIG. 2 is a side view of the distal portion of the catheter of FIG. 1;

FIG. 3 is an enlarged overall view of a section of the catheter of FIG. 2;

FIG. 4 shows the catheter of FIG. 2 with a release wire within a release wire lumen;

FIG. 5 is an enlarged overall view of a section of the catheter and release wire of FIG. 4;

FIG. 6 shows the structure of FIG. 5 with a generally helical covered stent mounted thereto in a radially contracted, first state, the structures constituting the distal portion of the coiled stent delivery assembly of FIG. 1, the target location within the body lumen being indicated by dashed lines;

FIG. 6A is an enlarged view of the covered stent of FIG. 6 showing the release wire piercing the distal end of the covered stent;

FIG. 7 illustrates the assembly of FIG. 6 after the release wire has begun to be retracted to release the distal portion of the stent from the catheter;

FIG. 8 shows the assembly of FIG. 7 after the release wire has been retracted further to release part of the intermediate portion of the stent;

FIG. 9 shows the assembly of FIG. 8 after the release wire has been completely retracted and the covered stent is in a radially expanded, second state;

FIG. 10 shows the covered stent of FIG. 9 within the blood vessel and after the catheter has been removed;

FIG. 11 illustrates an alternative embodiment similar to the catheter of FIG. 3 in which the catheter comprises separate tubes connected to one another;

FIG. 12 is an end of view of the catheter of FIG. 11;

FIG. 13 is another alternative embodiment similar to the catheter of FIG. 3 in which the catheter lacks the cutouts of the FIG. 3 embodiment but rather has perforations extending into the release wire lumen, the perforations acting as the entrances and exits of the lumen segments;

FIG. 14 illustrates the embodiment of FIG. 13 in which the release wire passes through the perforations in a weaving pattern;

FIG. 15 illustrates a still further alternative embodiment including a modified release wire assembly similar to the release wire assembly of FIG. 1A and a modified catheter similar to the catheter of FIG. 5;

FIGS. 16 and 17 are cross-sectional views of the catheter taken along lines 16-16 and 17-17 in FIG. 15 illustrating the presence of a distal release wire in FIG. 16 and both the distal and a proximal release wire in FIG. 17;

FIG. 18 is an overall view of the release wire assembly of FIG. 15 showing the use of distal and proximal release wires;

FIG. 19 illustrates the result of initially pulling on the release wire assembly of FIG. 15 causing the proximal release wire to disengage from the proximal end of the covered stent;

FIG. 20 illustrates the result of continuing to pull on the release wire assembly of FIG. 19 causing the distal release wire to disengage from the distal end of the covered stent, after which continued pulling on the release wire assembly will cause the distal release wire to release the intermediate portion of the covered stent to assume the radially expanded, second state of FIGS. 9 and 10;

FIG. 21 is a simplified enlarged side elevational view of the catheter of FIG. 2 at the distal cut out illustrating the use of a braided material embedded within a polymer to form the outer surface of the catheter to provide additional torsional strength;

FIG. 22 is a simplified cross-sectional view taken along line 22-22 of FIG. 21;

FIG. 23 is an overall view of the distal portion of a further embodiment of the invention comprising a coiled endoluminal prosthesis delivery assembly having a coiled endoluminal prosthesis secured thereto in a radially contracted state;

FIG. 24 is an enlarged view of the distal and proximal ends of the assembly of FIG. 23;

FIG. 25 illustrates the distal portion of the delivery catheter of the embodiment of FIG. 23 in a relaxed state with the coiled endoluminal prosthesis and the release wire removed to illustrate a second, metal tube mounted to the distal end of a first, flexible, polymer based tube, the Fig. also showing the spiral circumferential offset of the release wire guide tubes mounted to the outer surface of the second, metal tube, and flexibility-enhancing relief areas created along the length of the second, metal tube;

FIG. 26 is a simplified cross-sectional view taken along line 26-26 of FIG. 25 showing a guide tube mounted to the outside surface of the second, metal tube;

FIG. 27 is a partial cross-sectional view taken along the axis of the second, metal tube showing openings formed through the tubular wall of the second, metal tube to create the relief areas along the second, metal tube;

FIG. 28 illustrates the structure of FIG. 25 after the second, metal tube has been placed in a torqued state, as indicated by the arrows at either end of the second, metal tube, so that the guide tubes become generally axially aligned, whereby after the coiled endoluminal prosthesis of FIG. 23 has been mounted to the delivery catheter, the second, metal tube tends to tighten the coiled endoluminal prosthesis onto the second, metal tube thereby helping to create a smaller placement profile by reducing the cross-sectional size of the delivery catheter;

FIGS. 29-35 are directed to a further aspect of the invention in which the coiled endoluminal prosthesis assembly includes a handle that can be operated to simultaneously rotate the delivery catheter and retrieve the release wire;

FIG. 29 shows a handle from which the delivery catheter extends with a portion of the handle body broken away to show how the release element is wound about a spool as the delivery catheter is rotated by a rotator knob;

FIG. 30 is an exploded isometric view of the structure of FIG. 29;

FIG. 31 illustrates an alternative embodiment to the handle of FIGS. 29 and 30 in which the release wire is pulled axially by a grooved follower sleeve as the delivery catheter is rotated;

FIG. 32 is a view of an embodiment similar to that of FIG. 31 but in which grooved follower sleeve is much shorter than the embodiment of FIG. 31 by the use of multiple spiral groove pins carried by the handle body;

FIGS. 33-35 illustrate a still further alternative embodiment of the handle of FIGS. 29 and 30 similar to the embodiment of FIG. 32 but having a continuous internal thread engaging the spiral groove in the grooved follower sleeve and also having a pull ring assembly that can be used to rotate the catheter and withdraw the release wire more rapidly than would typically occur by turning the rotator knob;

FIGS. 36-40 illustrate a sequence of releasing a constant length endoluminal prosthesis from a delivery catheter;

FIG. 41 illustrates the distal end of a delivery catheter similar to that of FIG. 4 but having a balloon mounted along its length between two cutouts; and

FIG. 42 shows the delivery catheter of FIG. 41 with a constant length endoluminal prosthesis, similar to that shown in FIGS. 36-40, mounted thereto.

### DETAILED DESCRIPTION OF THE INVENTION

The present invention will be described with reference to several embodiments with like reference numerals referring to like elements. The following description of the invention will typically be with reference to specific structural embodiments and methods. It is to be understood that there is no intention to limit the invention to the specifically disclosed embodiments but that the invention may be practiced using other features, elements, methods and embodiments.

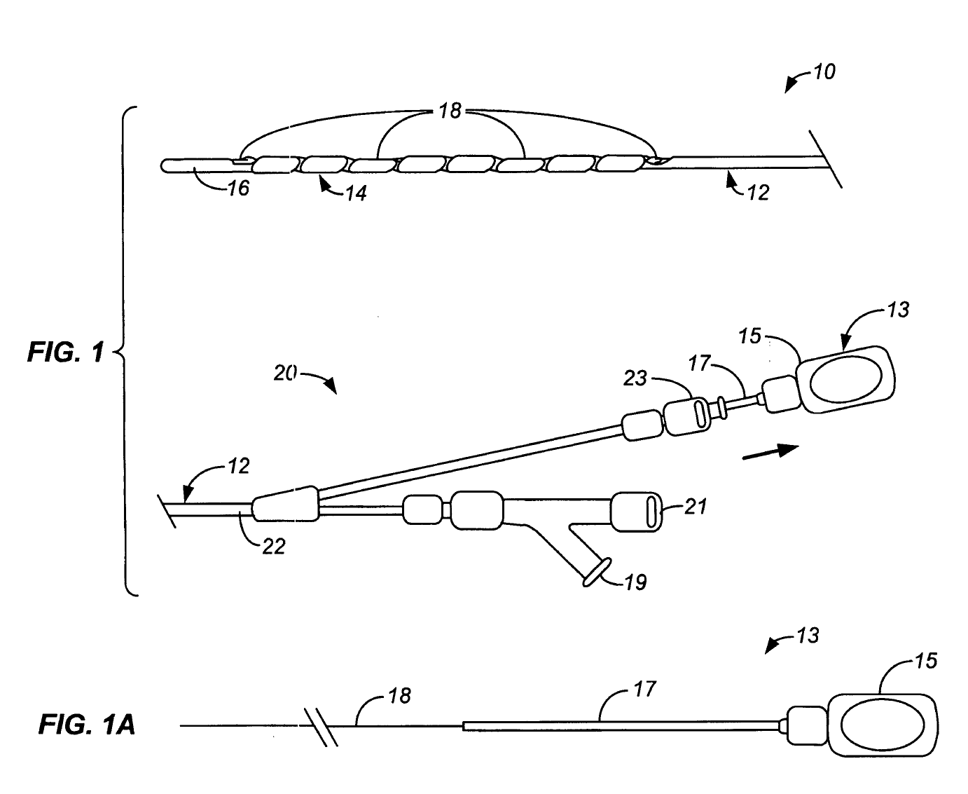

FIG. 1 is an overall view of a coiled stent delivery assembly 10 made according to a first aspect of the invention. Assembly 10 is shown to include a catheter 12 with a generally helical covered stent 14 mounted to the distal portion 16 of catheter 12. Covered stent 14 may be of the type disclosed in U.S. Pat. No. 6,572,648 or 6,645,237 including a ladder type stent covered by a graft material. Assembly 10 also includes a release wire assembly 13, see FIG. 1A, including a flexible release wire 18, extending along catheter 12, connected to a finger grip 15 by a relatively rigid tube 17. Assembly 10 further includes a proximal end assembly 20 secured to the proximal end 22 of catheter 12. Proximal and assembly 20 includes various fittings and ports, such as flush port 19, guide wire port 21 and release wire port 23. The construction of the various components of assembly 10 will now be described.

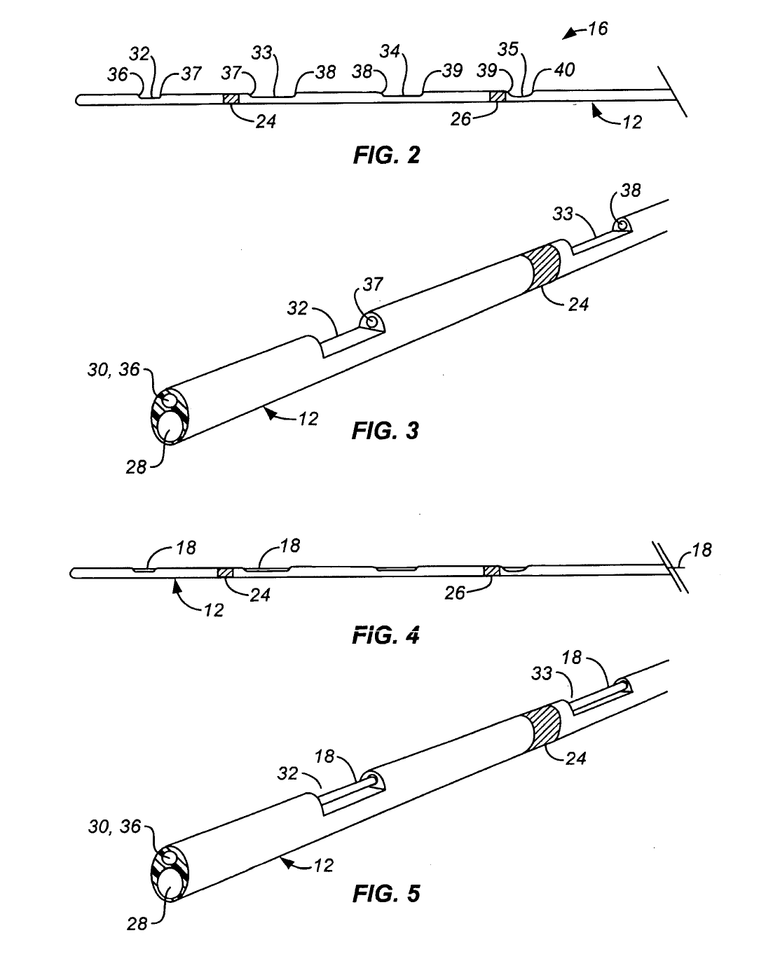

FIG. 2 is a side view of the distal portion 16 of catheter 10 while FIG. 3 is an enlarged overall view of a section of distal portion 16. Distal portion 16 has a pair of radiopaque markers 24, 26 used to help properly position covered sent 14 within the body lumen. Catheter 12 has a main lumen 28 and a release wire lumen 30. Main lumen 28 is typically used for passage of a guide wire and may also be used for other purposes, such as irrigation, aspiration, passage of tissue extraction devices, and so forth. FIGS. 4 and 5 show release wire 18 within release wire lumen 30.

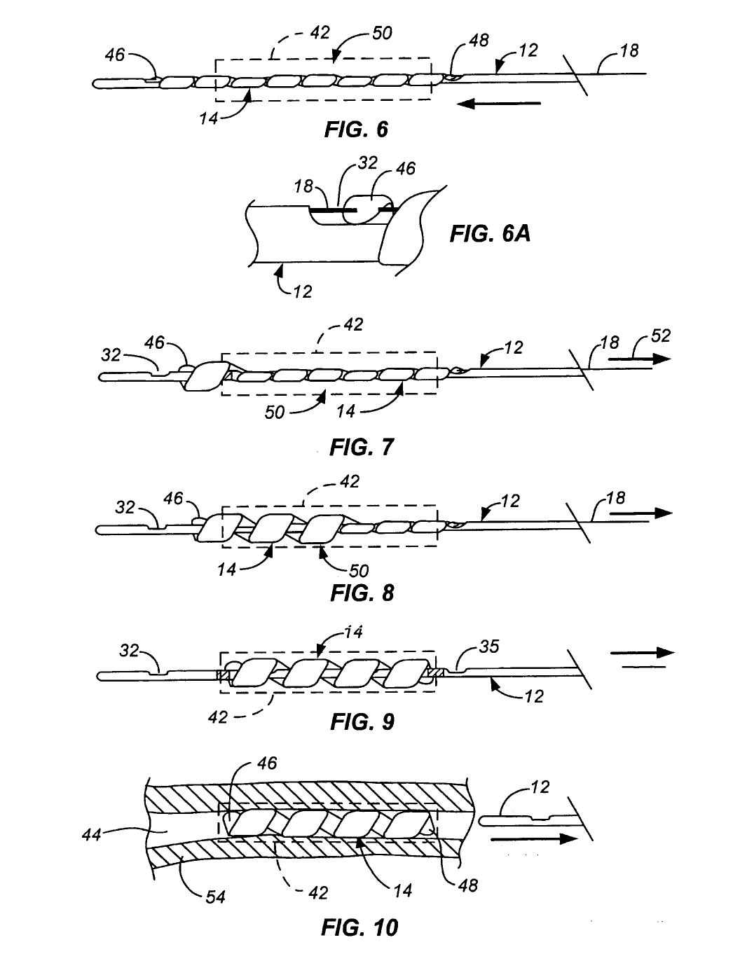

Distal portion 16 of catheter 12 has a number of axially spaced apart cutouts 32, 33, 34 and 35, which create a series of lumen segments 36, 37, 38, 39 and 40 separated by cutouts 32-35. Accordingly, release wire 18 passes along a release wire path including internal release wire path segments defined by lumen segments 36-40 and external release wire path segments along cutouts 32-35. That is, the external release wire path segments extend between the exit of one lumen segment and the entrance of an adjacent lumen segment. FIG. 6 shows covered stent 14 mounted to distal portion 16 of catheter 12 in a radially contracted, first state. Note that covered stent 14 is considered positioned within a body lumen 44 defined by a blood vessel 54, or other hollow body structure, in FIGS. 6-10 but, for simplicity of illustration, blood vessel 54 is only shown in FIG. 10. The target location 42 within the body lumen 44 of blood vessel 54 is indicated by dashed lines. The distal and proximal ends 46 and 48 of covered sent 14 are secured to distal portion 16 of catheter 12 by release wire 18 passing through distal end 46 at cutout 32 as shown in FIG. 6A and through proximal end 48 at cutout 35. The intermediate portion 50 of covered stent 14 is secured to distal portion 16 of catheter 12 at two positions, that is at intermediate cutouts 33, 34, by passing between release wire 18 and catheter 12 and the intermediate cutouts.

The longitudinal or axial length of cutouts 32-35 is oversized with respect to covered stent 14 housed therein. It has been found that making cutouts 32-35, and especially intermediate cutouts 33 and 34, oversized helps to prevent damage to covered stent 14 during assembly and use. In one embodiment catheter 12 has an outside diameter of 1.5 mm (0.060 in.), main lumen 28 has an inside diameter of 1 mm (0.037 in.), release wire 18 has a diameter of 0.3 mm (0.012 in.), and each turn of covered stent 14 when wrapped down as shown in FIG. 1 has an axial or longitudinal length of about 5 or 6 mm. It has been found that making cutouts 33 and 34 about 8 mm, that is about 2 mm longer than the axial or longitudinal length of covered stent 14, helps to eliminate binding of and damage to the covered stent. The proximal and distal cutouts 35 and 32 are, in one embodiment, each about 4 mm long. The space between the cutouts is, in this embodiment, 11 mm long to permit two turns of covered stent 14 between each cutout. Some overlap of the turns of covered sent 14 over adjacent cutouts does not compromise the functionality of the catheter. Cutouts 32-35 are shown in FIG. 3 having a flat bottom; the cutouts may also be made with, for example, a convex bottom surface.

Assembly 10 is positioned at target location 42 while in the wound down, radially contracted, first state of FIGS. 1 and 6. Proper positioning of covered stent 14 within blood vessel 54 is aided by the use of radiopaque markers 24, 26. FIG. 7 illustrates release of distal end 46 of covered sent 14 by pulling on release wire 18 as indicated by arrow 52. An undesirable, uncontrolled expansion of covered stent 14, sometimes referred to as a “jack-in-the-box” release, could occur on release of one of the distal and proximal ends 46, 48 of covered stent 14 if it were only secured at its ends. Such a “jack-in-the-box” release is not desired because it can adversely affect the proper final positioning of covered stent 14. A “jack-in-the-box” release is avoided in this embodiment by the provision of intermediate cutout 33, 34 to permit covered stent 14 to be placed between release wire 18 and catheter 12 at the intermediate cutouts.

FIG. 8 illustrates the result of continuing to pull release wire 18 causing release wire 18 to be removed from intermediate cutout 33 thereby releasing a part of intermediate portion 50. FIG. 9 illustrates covered stent 14 within its radially expanded, second state after release wire 18 has been completely removed from catheter 12, that is after removing release wire 18 from intermediate cutout 34 and from proximal end 48 of covered stent 14 at proximal cutout 35. The present invention provides a very controlled release of covered stent 14 to help ensure its proper placement within body lumen 44. FIG. 10 shows covered stent 14 fully expanded within body lumen 44 of blood vessel 54 and after catheter 12 has been removed from blood vessel 54.

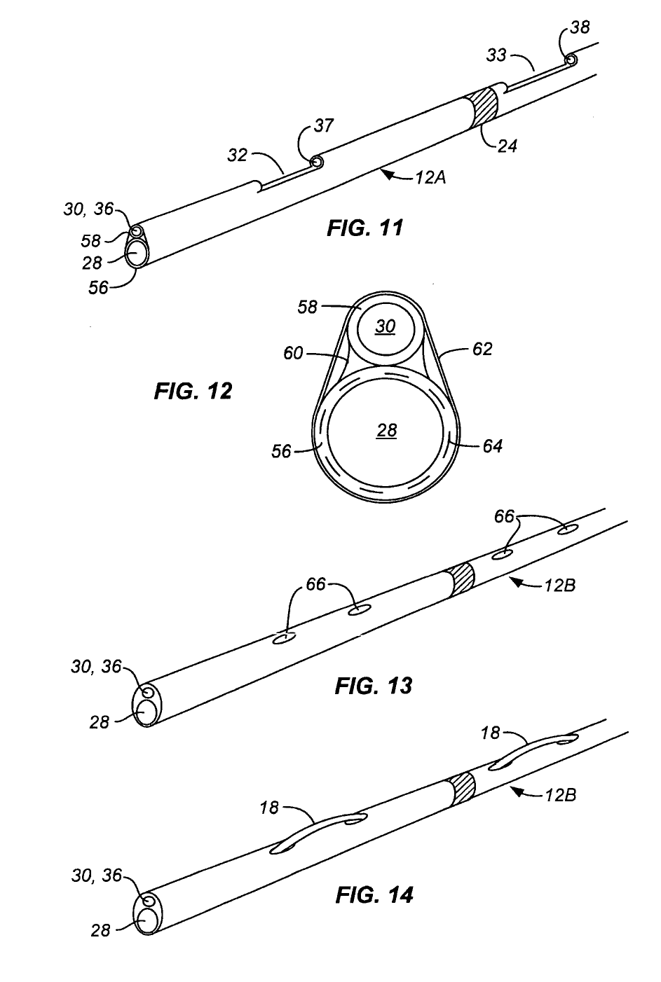

FIGS. 11 and 12 illustrate an alternative embodiment similar to catheter 12 of FIG. 3. Catheter 12A comprises first and second tubes 56, 58 connected to one another by adhesive 60 and heat shrink tubing 62. First tube 56 preferably has stainless steel flat wire braid filaments 64 to enhance structural integrity.

Another alternative embodiment, similar to the catheter of FIG. 3, is shown in FIG. 13. Catheter 12B lacks the cutouts 32-35 of the catheter of FIG. 3 but rather has perforations 66 extending into release wire lumen 30, the perforations acting as the entrances and exits of the lumen segments. FIG. 14 shows catheter 12B with release wire 18 passing through perforations 66 in a weaving pattern so that perforations 66 act as the entrances and exits of lumen segments 36, 37 and 38 in this Fig. In some embodiments it may be desired to provide for a much greater number of perforations 66 than would be expected to be used, for example 20 or 40 perforations instead of 8. This would allow greater flexibility in the placement of the turns of covered stent 14 as well as the number of turns to be captured between release wire 18 and sent 12. If the catheter were made from a porous material, the pores in the material itself may provide the perforations. Also, the perforations could also be formed in the catheter using a tool as the covered stent is wound onto the catheter; such a tool could also used to help to guide the release wire out through or into the newly formed perforation or both out through and into the newly formed perforation.

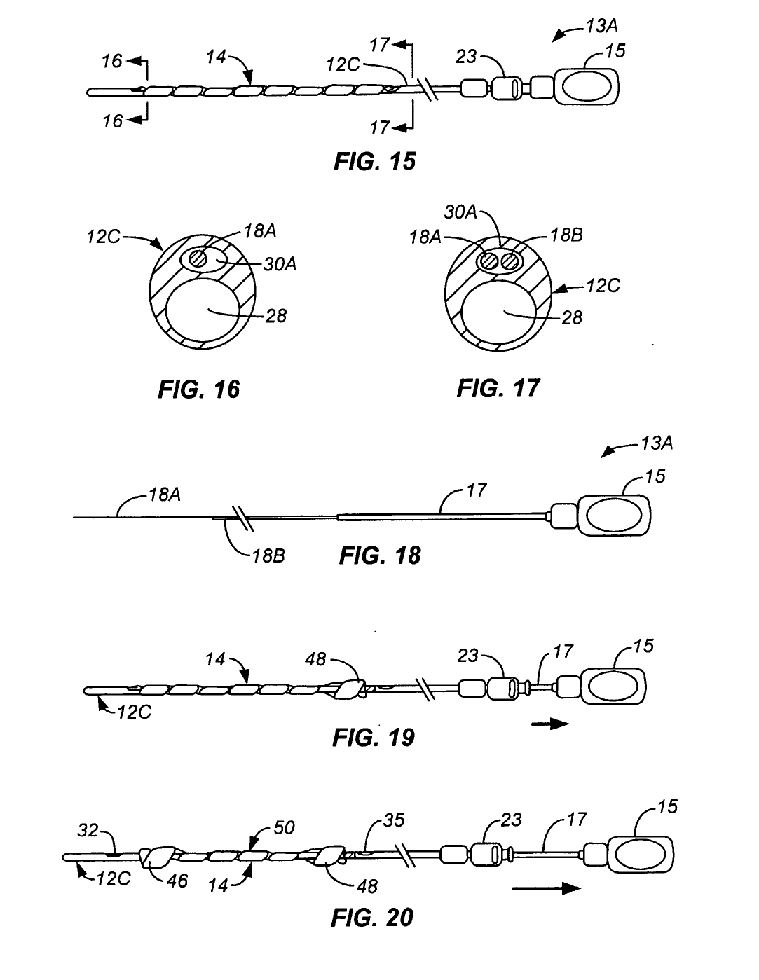

FIG. 15 illustrates aspects of a still further alternative embodiment of the present invention. A modified release wire assembly 13A, shown best in FIG. 18, is used with a modified catheter 12C. FIGS. 16 and 17 are cross-sectional views of catheter 12C showing the presence of an oval or other other-than-round release wire lumen 30A. Release wire lumen 30A is sized and shaped to house both distal and proximal release wires 18A and 18B. If more than two release wires were to be used, the release wire lumen can be appropriately sized and shaped. It also may be desirable to use an other-than-round cross-sectional shape for the release wire for greater space utilization.

FIG. 18 is an overall view of the release wire assembly of FIG. 15 showing the use of distal and proximal release wires 18A and 18B. In this embodiment the release wires are pulled simultaneously by finger grip 15. If desired, release wires could be manipulated individually. FIG. 19 illustrates the result of initially pulling on the release wire assembly of FIG. 15. In this embodiment the length of proximal release wire 18B is chosen so that proximal end 48 of covered sent 14 is released first. FIG. 20 illustrates the result of continuing to pull on release wire assembly 13A causing distal release wire 18A to disengage from distal end 46 of the covered stent. Continued pulling on release wire assembly 13A will cause distal release wire 18A to release intermediate portion 50 of covered stent 14 to assume the radially expanded, second state of FIGS. 9 and 10. Instead of using individual release wires, such as distal and proximal release wires 18A and 18B, a single, main release wire can be used having release wire side branches welded or otherwise secured to the main release wire; the release side wire branches would then be the used to engage covered stent 14 and various positions along the covered stent. The lengths of the release wire side branches and the main release wire can be chosen to permit release of covered stent 14 from cutouts 32-35 in any order desired, including from proximal cutout 35 to intermediate cutout 34, to intermediate cutout 33 and finally to distal cutout 32. Such a release from the proximal cutout 35 to the distal cutout 32 could also be accomplished in other manners, such as my extending the release wire to the distal end of the catheter, reversing direction, and then directing the release wire along the release wire lumen back towards the proximal end of the catheter.

Instead of the release schemes discussed above, other release schemes can be used. For example, release can start simultaneously at proximal end 48 and end at distal end 46; also, release of covered stent 14 can be from one or both of intermediate cutouts 34 and then from one end and then from the other end. The number and spacing of the cutouts and perforations can also be changed. Whatever release scheme is to be used, in some embodiments it is preferred that at most 50%, and more preferably at most 25%, of covered sent 14 simultaneously move to the radially expanded, second state in contact with blood vessel 54 or other hollow body structure. In one preferred embodiment, using 4 equally spaced cutouts, at most about 33% of the length of covered stent moves simultaneously to the radially expanded, second state.

The present invention has been described as using a release wire. The release wire is not limited to structures or materials which are commonly classified as wire, that is single or multiple strands of metal. Rather, release wire also includes threads or strands or other lengths of material which may or may not have significant flexural strength and may be nonmetallic or a combination of metallic and nonmetallic materials. The particular mechanical characteristics for the release wire will depend on the operating conditions, including, for example, the length of the cutouts, the force expected to be exerted by the covered stent when in the radially contracted, first state, and the number of release wires used.

The release wire and the associated release wire lumen and lumen openings in the catheter are used to engage the covered stent and maintain it in the radially contracted, first state and then control the subsequent releasing of various portions of the covered stent to prevent the sudden, undesirable “jack-in-the-box” deployment of the covered stent. Instead of a release wire, the covered stent may be retained in the radially contracted, first state using a heat softenable adhesive between the covered stent and the catheter. An appropriate source of heat can be used to selectively heat and thus soften the adhesive. The source of heat could be an RF device positionable at various locations along the main lumen or a number of individually operable resistance heating elements formed in the catheter. Another alternative to a release wire would be to tie the covered stent to the catheter using a loop of thread at each securement point; the loop of thread would pass into the main lumen, through the wall of the catheter, over or through the covered stent, back through the wall of the catheter and into the main lumen to complete the loop. The covered stent could then be released by withdrawing a thread cutter through the main lumen of the catheter causing the loops of thread to be cut, typically one at a time. Other structure and procedures may be used as a substitute for the disclosed release wire arrangement.

Various embodiments of the invention may and preferably do provide one or more of the following advantages: simplicity of design and ease-of-use, ability to release a coiled stent gradually, and accuracy of placement.

Other modification and variation can be made to the disclosed embodiments without departing from the subject of the invention as defined in following claims. For example, instead of providing a separate release wire lumen, in some embodiments the delivery catheter may include a single lumen through which the release wire passes; however, it is preferred that a separate release wire lumen be provided because having a separate release wire lumen helps to reduce the tendency of the release wire to bend so the release wire holds the covered stent more securely. Having a separate release wire lumen helps to prevent any interference with the passage of the guide wire or other devices through the catheter. In some situations it may not be necessary to provide distal lumen segment 36. For example, the distal end of release wire 18 could be releasably secured to distal end 46 of covered stent 14 by, for example, bending the distal end of the release wire (which would straighten when pulled), adhering the release wire to the distal end using an adhesive (which adhesive bond could be broken when the release wire was pulled), or a securing the release wire to the distal end by a breakable thread (which would break when the release wire was pulled). In the preferred embodiments the release wire engages the tips of the proximal and distal portions of the covered stent; in appropriate cases it may be possible or desirable to engage the covered stent at positions spaced apart from the tips of the proximal and distal portions. The invention has been described with reference to a covered stent. However, other generally helical endoluminal prostheses may also be used. For example, a bare metal stent or a metal stent coated with a polymer/drug matrix may be used. In the preferred embodiments the release wire passes through or pierces the proximal and distal ends of the covered stent while the intermediate portion of the covered stent passes between the release wire and the catheter; in some situations it may be desirable to have the release wire pierce one or more locations along the intermediate portion of the covered stent. While the stent is typically released by pulling on the release wire, release may also be accomplished in appropriate situations by pushing on the release wire.

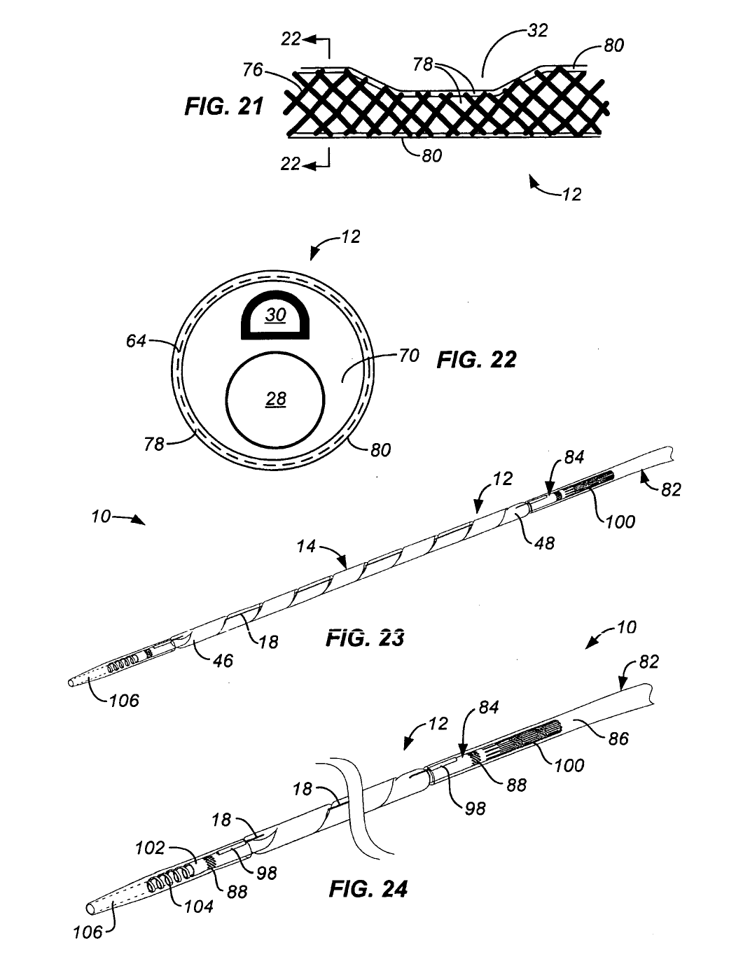

Use of a braided material including filaments of 64, see FIG. 12, to increase the torsional strength of a catheter is well-known. FIG. 21 is a simplified enlarged side elevational view of a modification of the notched catheter 12 of FIG. 2. Braid-reinforced notched catheter 12 of FIGS. 21 and 22 has a catheter body 70 including an outer surface including a generally cylindrical outer surface portion 72 and a notched surface portion 74. Next, a filament-containing layer, typically of braided material 76, is placed adjacent to surface portions 72, 74. Braided material 76 fits snugly against surface portions 72, 74 as indicated in FIG. 21. Next, a mandrel or other elongate element, not shown, is inserted through release wire lumen 37 to pierce braided material 76 as the mandrel exits lumen segment 37 and again as it enters lumen segment 36 to capture a portion of braided material 76 between notched surface portion 74 and the mandrel. Next, a polymer sleeve, typically made of nylon or some other polymer 78, is slid over braided material 76 and then heated to bond braided material 76 to catheter body 70. Thereafter polymer material 78 is removed from the mandrel to permit the mandrel to be removed from lumen 30. This results in a notched catheter having an effectively uninterrupted filament-containing layer 80 at its outer surface, even at cutouts 32-35. In this way the torsional strength provided by braided material 76 in filament-containing layer 80 is not compromised as it would be if the filament-containing layer were placed over the outer surface of catheter body 70 before the cutouts were made. Instead of a separately applied polymer material 78, braided material 76 and polymer material 78 could be provided as a pre-preg (previously impregnated) material.

The various coiled stent delivery assemblies 10 discussed above with reference to FIGS. 1-22 may be used in a manner by which catheter 12 is rotated simultaneously as release wire 18 is pulled or otherwise manipulated to release stent 14 from the catheter. FIGS. 23-28 disclose an assembly 10 in which the distal portion of delivery catheter 12 is flexible but has superior torsional stiffness. Delivery catheter 12 in this embodiment comprises a first, polymer-based tube 82 and a second, metal tube 84 secured to the distal end 86 of first tube 82. First tube 82 is preferably a braid-reinforced polymer while second tube 84 is typically a stainless steel hypotube having flexibility-enhancing relief areas 88 along the length of second tube 84. In the disclosed embodiment relief areas 88 comprise a series of angled openings 90 extending between the inside surface 92 and the outside surface 94 of the tubular wall 96 of second tube 84. While relief areas 88 are positioned at regular locations along second tube 84, they could be placed at irregular intervals or they could be located generally continuously along the entire length or portions of the entire length of second tube 84. Also, some or all of relief areas 88 need not extend completely through tubular wall 96.

FIG. 23 is an overall view of coiled endoluminal prosthesis delivery assembly 10 having a coiled endoluminal prosthesis, such as covered stent 14, secured to second, metal tube 84 in a radially contracted state through the use of release wire 18. Release wire 18 passes through a series of guide tubes 98 with turns of stent 14 passing over the guide tubes. As shown in FIGS. 23 and 24, turns of stent 14 are captured between release wire 18 and second, metal tube 84 in regions between adjacent guide tubes 98. The proximal end 100 of second, metal tube 84 is adhered within distal end 86 of first tube 82. The distal end 102 of second tube 84 comprises a highly flexible coil 104 and is covered by in atraumatic covering 106.

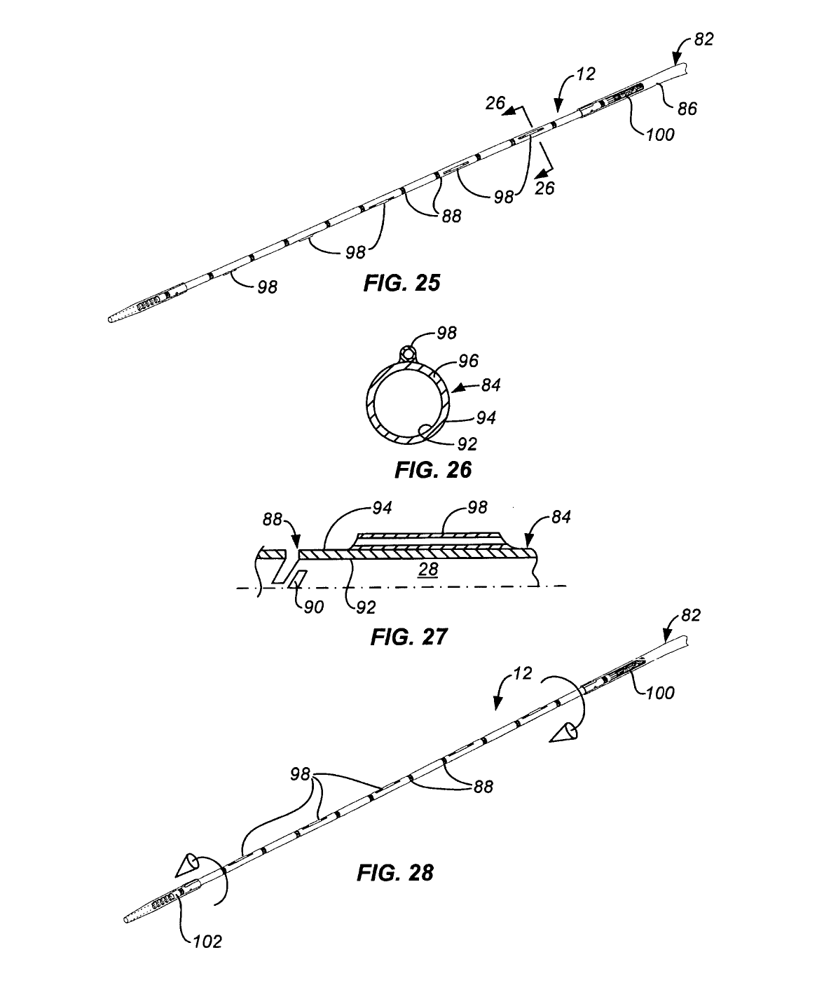

FIG. 25 illustrates second, metal tube 84. It can be seen that guide tubes 98 have a left-hand or counterclockwise spiral circumferential offset when viewed from proximal end 100 of second tube 84. Prior to mounting stent 14 onto second tube 84, second tube 84 is placed in a torqued state, as indicated in FIG. 28, by twisting distal end 102 of second tube 84 in a right hand or clockwise circumferential direction when viewed from proximal end 100. This causes guide tubes 98 to become generally axially aligned. After covered stent 14 has been mounted to second tube 84, second, metal tube is released and tends to tighten the covered stent onto the second, metal tube thereby helping to create a smaller placement profile by reducing the cross-sectional size of the delivery catheter. Pre-torquing second tube 84 accommodates variations in the length of stent 14 and in the placement of stent 14 on second tube 84 so to facilitate proper alignment of distal end 46 of covered stent 14 with the distal-most guide tube 98.

Using a flexible metal tube as the portion of catheter 12 on which covered stent 14 is mounted provides several advantages in addition to enhanced torsional strength. The wall of the metal tube can be thinner for the same torsional strength so that main lumen 28 can be larger to permit the use of a larger diameter guide wire compared with polymer-based guide catheters. Metal tube 84 will typically have a smaller profile (smaller cross-sectional diameter) than a polymer-based catheter having an equivalent torsional strength. Also, metal tube 84 can remain in a pre-torqued state for a much longer time than an equivalent polymer-based catheter for greatly enhanced storage life. This permits assembly 10 to be shipped and stored in a wound-down state and not have any appreciable effect on second tube 84.

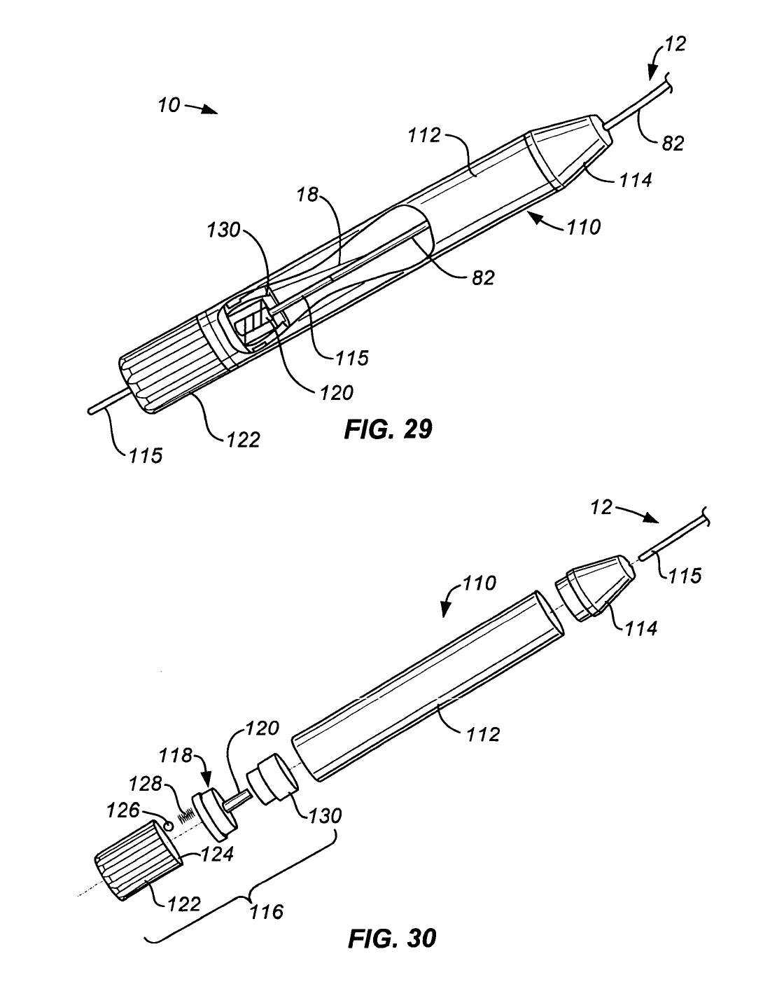

When a generally helically wound coiled stent moves from the radially contracted state to the radially expanded state, see FIGS. 6-10, the number of coils decreases as the stent expands resulting in circumferential movement of the stent. A further aspect of the invention is the recognition that as covered stent 14, or some other endoluminal prosthesis, is released into a blood vessel or other hollow body organ, typically starting from one end of the covered stent and proceeding towards the other end of the covered stent, it would be advantageous to simultaneously rotate catheter 12. Doing so would help keep the coiled stent in place so that it rolls out against the wall of the hollow body organ in a controlled manner. While this could be accomplished by manually rotating catheter 12 as release wire 18 is pulled, more control may be achieved using, for example, one of the handles shown in FIGS. 29-35. These figures illustrate several embodiments of handles that can be operated to simultaneously rotate catheter 12 and retrieve release wire 18.

FIGS. 29 and 30 show a handle 110 from which delivery catheter 12 extends. Handle 110 comprises a handle body 112 to which a nose piece 114 is secured. Catheter 12 comprises first polymer-based tube 82 to which a stiff metal hypotube extension 115 is mounted at its distal end. Handle 110 also includes a release element retractor and catheter rotator assembly 116. Assembly 116 includes a spool device 118 secured to handle body 112, device 118 comprising an axially extending spool 120 to which release wire 18 is wound. Assembly 116 also includes a rotator knob 122 mounted adjacent to spool device 118 and secured to hypotube extension 115 so that rotating rotator knob 122 causes catheter 12 to rotate. Rotator knob 122 has a ring of depressions or detents 124 engageable by a ball 126, ball 126 being biased toward detents 124 by a spring 128. Ball 126 and spring 128 are captured between rotator knob 122 and spool device 118. Assembly 116 also includes a release element guide 130 affixed to hypotube extension 115. Release wire 18 is secured to spool device 118 and passes through an opening (not shown) in release element guide 130 so that rotating rotator knob 122 relative to handle body 112 also rotates release element guide 130 and causes release wire 18 to be wound onto or off of spool 120. The speed of retrieval of release wire 18 relative to the rotation of delivery catheter 12 can be changed by changing the diameter of spool 120. If desired, handle 110 could be modified to prevent, for example, unwinding release wire 18 from spool 120 or to prevent inadvertent rotation of rotator knob 122 relative to handle body 112. While delivery catheter 12 in this embodiment is manually rotated, handle 110 could be modified to provide for motorized rotation of the delivery catheter with the simultaneous retrieval of release wire 18. The invention may also be carried out, in appropriate circumstances, by the simultaneous rotation of delivery catheter 12 and the unwinding of release wire 18 from spool 120.

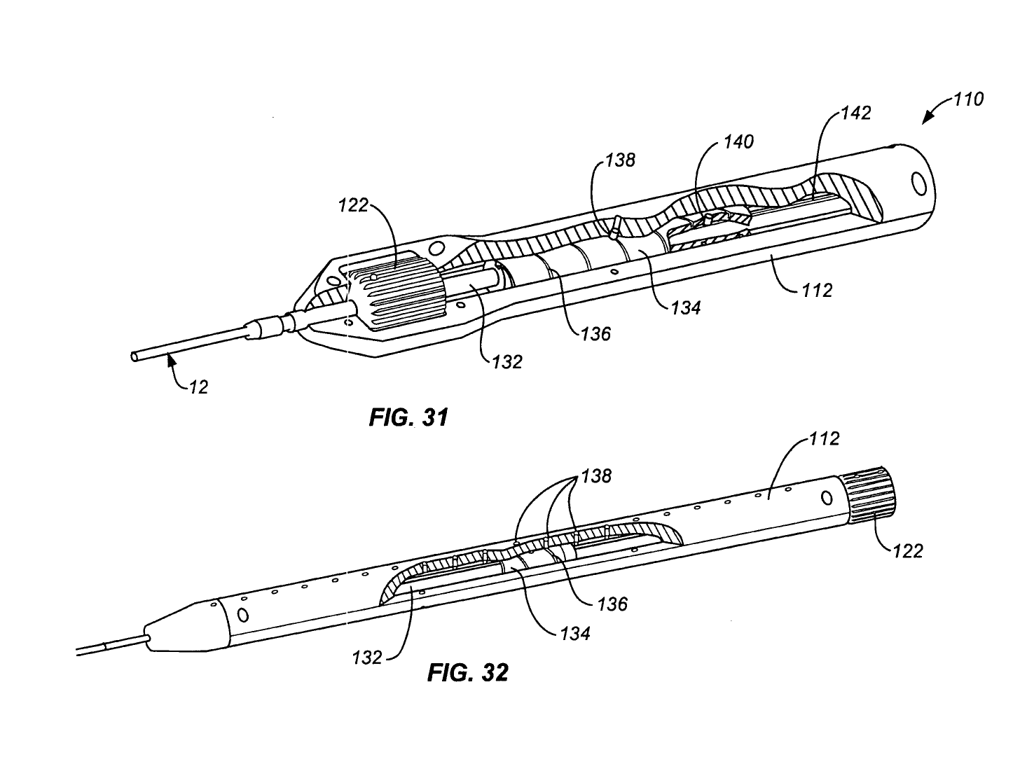

FIG. 31 illustrates an alternative embodiment to handle 110 of FIGS. 29 and 30. Catheter 12 is fixed to rotator 122 so that rotating rotator 122 causes the catheter to rotate relative to handle body 112. A tubular rotator extension 132 extends proximally from rotator 122 within handle body 112. A grooved follower sleeve 134 is positioned between the interior wall of handle body 112 and rotator extension 132. Sleeve 134 has a spiral groove 136 on its outer surface. A spiral groove pin 138 extends from a fixed position along handle body 112 to engage spiral groove 136. A pull wire connection element 140 is carried by sleeve 134 and extends radially inwardly to pass through an axially-extending slot 142 in rotator extension 132. Release wire 18, not shown in FIG. 31, passes through the hollow interior of rotator extension 132 and is secured to pull wire connection element 140. Therefore, rotating rotator extension 132 relative to handle body 112 causes rotator extension 132 to rotate; the rotation of rotator 122 causes slot 142 to rotate thus causing sleeve 134 to rotate through the engagement of pull wire connection element 140 within slot 142. However, the engagement of pin 138 within spiral groove 136 causes sleeve 134 to move axially as it rotates. This results in the rotation of catheter 12 and, typically, the pulling of release wire 18 when rotator 122 is rotated.

FIG. 32 is a view of an embodiment similar to that of FIG. 31 but in which grooved follower sleeve 134 is much shorter than in the embodiment of FIG. 31. This is possible through the use of multiple spiral groove pins 138 carried by handle body 112.

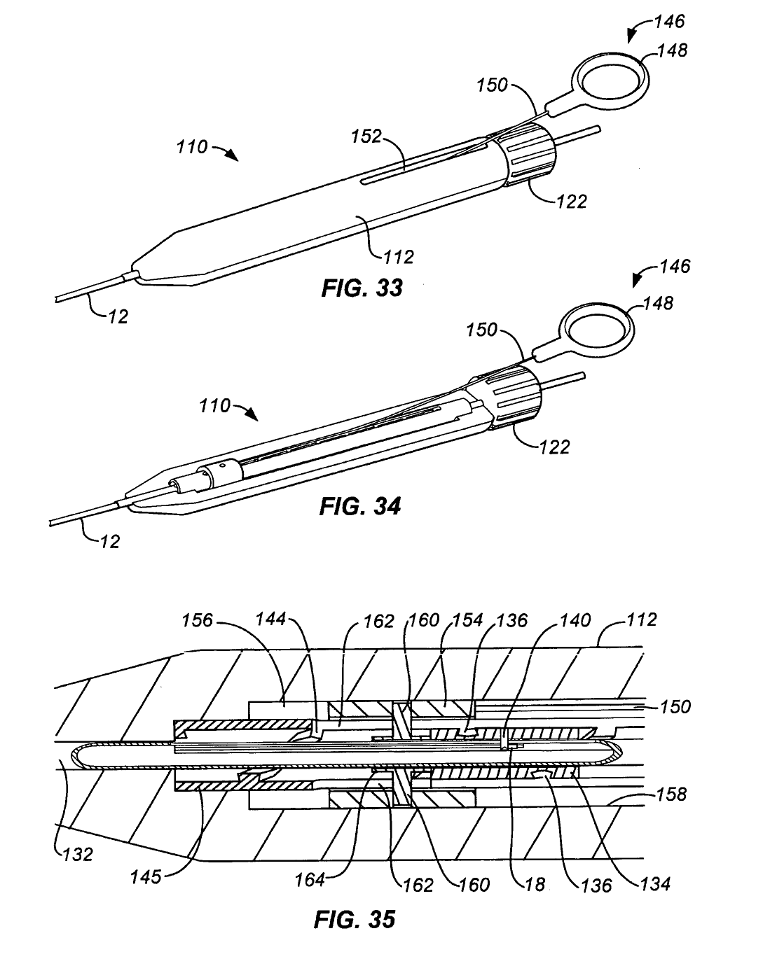

FIGS. and 33-35 illustrates a still further alternative embodiment of the handle of FIGS. 29 and 30 similar to the embodiment of FIG. 32 but differing in several respects. First, the embodiment of FIGS. 33-35 has a continuous internal thread 144 extending inwardly from a fixed (relative to handle body 112) thread structure 145 to engage spiral groove 136 in grooved follower sleeve 134. The second major distinction is the use of a pull ring assembly 146 that can be used to rotate catheter 12 and withdraw release wire 18 more rapidly than would typically occur by turning rotator knob 122. Assembly 146 comprises a pull ring 148 connected to a pull wire 150. Pull wire 150 extends through a slot 152 in handle body 112 and connects to a cylindrical slider 154. Slider 154 slides along an annular space 156 defined between a cylindrical inner wall 158 of handle body 112 and thread structure 145. Slider 154 has a pair of radially inwardly extending pins 160, the pins passing through slots 162 formed through opposite sides of thread structure 145. Pins 160 are secured to a pusher sleeve 164 that passes over rotator extension 132 and abuts follower sleeve 134. Therefore, pulling on pull ring 146 causes pusher sleeve 164 to drive grooved follower sleeve 134 proximally causing connector element 142 to pull release wire 18. Pulling on pull ring 148 also causes sleeve 134 to rotate, by the engagement of internal thread 144 and spiral groove 136, thereby rotating rotator extension 132 and catheter 12.

It can be appreciated that the embodiment of FIGS. 29 and 30 may be preferred over the embodiments of FIGS. 31-35 when it is useful or important to reduce the length of handle 110.

Structure other than release wire 18 could be used to maintain stent 14 in the wrapped down state. For example, individual constraining elements, such as loops or bands, could be used along catheter 12 to secure stent 14 to the catheter. A separate electric wire could be connected to each constraining element and energized to release each constraining element, and thus a portion of stent 14, in a desired order. Also, a single electric wire could be connected to all of the constraining elements with each constraining element being releasable, such as by melting a portion of the constraining element, after different periods of time. In both of these situations no movement of any release element would necessarily be required.

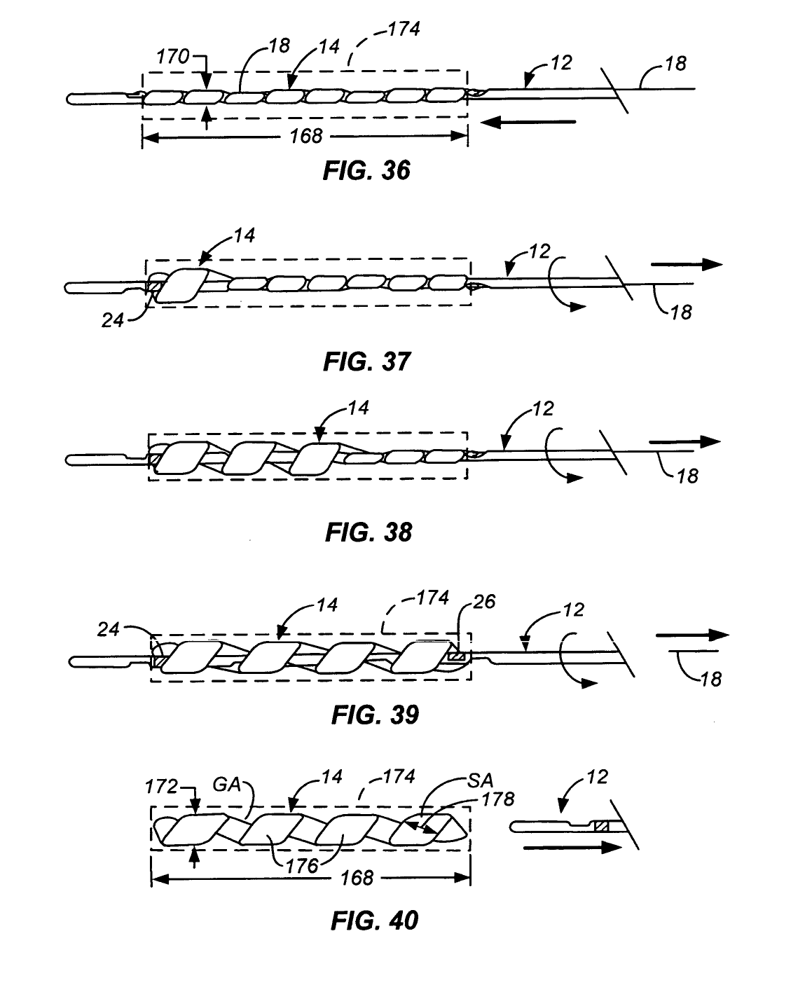

Another aspect of the invention is the recognition that it would be desirable if covered stent 14, or other helically wound endoluminal prosthesis, were to have the same length 168 when in a radially contracted state, see FIG. 36, as when in a relaxed, radially expanded state, see FIG. 40. By doing this, gross axial motions of covered stent 14 when moving from a radially contracted to a radially expanded state can being eliminated while minimizing the axial motion at any particular position along covered stent 14. FIGS. 36-40 illustrate a sequence of releasing a constant length covered stent 14 from delivery catheter 12. First, a first diameter 170 for covered stent 14 in a reduced diameter state is determined. Next, a second diameter 172, see FIG. 40, for coiled stent 14 when in an expanded diameter state at a target location 174 is determined. Covered stent 14 is configured to reduce or eliminate any difference between the lengths 168, 172 of covered stent 14 when in the reduced diameter state of FIG. 36 and when in the expanded diameter state of FIG. 40. One way to do is as follows. Assume covered stent 14, when in the expanded diameter state of FIG. 40, has a total area (TA) equal to the external surface area of the turns 176 of the endoluminal prosthesis (SA) plus the area of the generally helical gap (GA) between the turns. Covered stent of 14 is designed to reduce or eliminate any difference between the ratio of SA to TA to the ratio of first diameter 170 to second diameter 172. This is achieved by adjusting how closely turns 176 of covered stent 14 are wound down onto catheter 12.

One constraint on winding down covered stent is the desire not to have turns 176 lie on top of one another, and especially not have the solid stent portions of a covered stent lie on top of one another, so to limit any increase in cross-sectional area during stent placement. This may require adjusting the width 178 of covered stent 14 to prevent overlapping of turns 176 in a reduced diameter, wrapped down state, such as in FIG. 36. For example, instead of using a fixed width ladder type stent, such as shown in U.S. Pat. No. 6,488,700, the stent could have two side rails connected at their ends but without any rung elements to permit the width of the stent to be narrowed during wrap down if necessary. Also, a ladder type stent could have rungs that are bowed or otherwise act as flexible connectors to permit the side rails to be oriented closer to one another during wrap down onto the catheter. After release from the catheter, the stent would spring back to its relaxed width when the flexible connectors act as spring elements. In addition, width 178 of covered stent 14 could be chosen to prevent overlapping of turns 176.

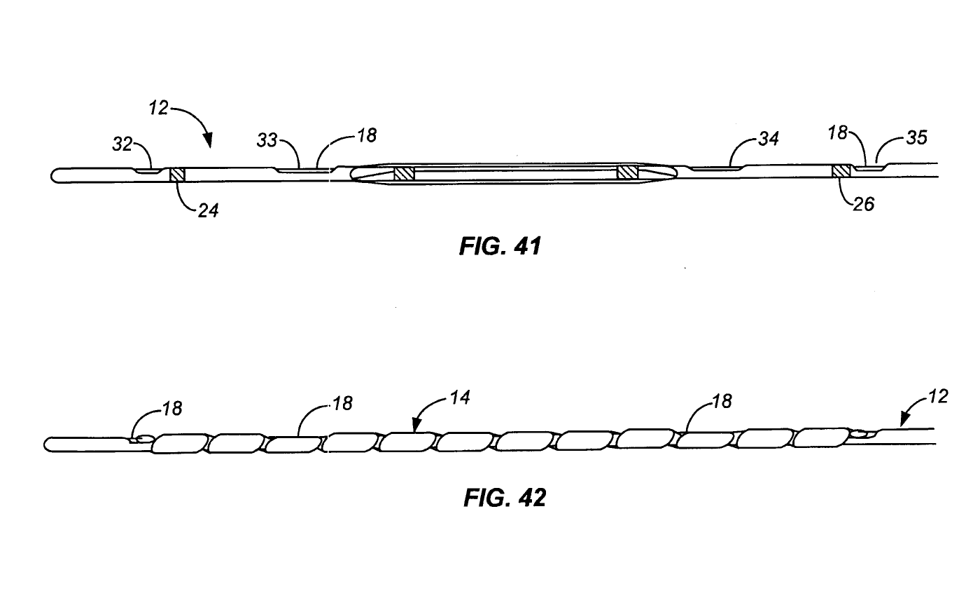

Another advantage, in addition to reducing or eliminating gross movements of portions of covered stent 14 during deployment, resulting from the use of a constant length covered stent 14 is that it permits the use of a balloon 180, see FIG. 41, to deploy or help deploy a helically coiled endoluminal prosthesis, such as covered stent 14. Balloon 180 is mounted to and carried by catheter 12 between cutouts 33, 34 after which generally helical covered stent 14 is mounted over catheter 12 and balloon 180 as shown in FIG. 42. Because of the minimal axial shifting of points along covered stent 14 during deployment, balloon 180 may be expanded before the entire covered stent 14 has been released while not significantly affecting any subsequent expansion movement by covered stent 14. While balloon 180 has been shown as being centrally positioned beneath covered stent 14, the balloon could be positioned elsewhere, such as at one end. Also, a number of balloons could be used.

Any and all patents, patent applications and printed publications referred to above are incorporated by reference.

The invention claimed is:

1. A notched catheter comprising: a catheter body having an outer surface and a longitudinally extending lumen; the outer surface having a main surface portion and a notched surface portion, the notched surface portion extending from the main surface portion to intersect the lumen; and a filament-containing layer adjacent to the main surface portion and the notched surface portion, whereby a portion of the filament-containing layer is capturable between the notched surface portion and an elongate element extendable through the lumen.

2. The notched catheter according to claim 2 wherein catheter body comprises a second, main lumen and the filament-containing layer comprises a braided material impregnated with a polymer.
